(19) United States
(12) Patent Application Publication
(10) Pub. No.: US 2025/0021623 A1
(43) Pub. Date: Jan. 16, 2025

(54) INFORMATION PROCESSING APPARATUS, INFORMATION PROCESSING METHOD, AND COMPUTER READABLE MEDIUM

(71) Applicant: Asahi Kasei Microdevices Corporation, Tokyo (JP)

(72) Inventors: Keiichiro KUWATA, Tokyo (JP); Takaaki FURUYA, Tokyo (JP)

(21) Appl. No.: 18/398,093

(22) Filed: Dec. 27, 2023

(30) Foreign Application Priority Data
Jan. 10, 2023 (JP) 2023-001892

(51) Int. Cl. G06F 17/18; G06F 21/44

(52) U.S. Cl. CPC G06F 17/18; G06F 21/44

(57) ABSTRACT

An information processing apparatus includes: a communication unit which receives statistical information transmitted from a sensor, the sensor having a measuring unit which measures environment information regarding environment in a target space and a statistics generation unit which generates, based on the environment information, the statistical information regarding the environment of the target space, a derivation unit which derives, based on the statistical information, a comfort index representing a comfort level of the target space according to a predetermined derivation method, and a generation unit which generates comfort information representing the comfort index of the target space.

The contents of the following patent application(s) are incorporated herein by reference:
NO. 2023-001892 filed in JP on Jan. 10, 2023

### BACKGROUND

### 1. Technical Field

The present invention relates to an information processing apparatus, an information processing method, and a computer readable medium.

### 2. Related Art

Patent document 1 discloses an apparatus which acquires a zoned real-time gas concentration and position information from a plurality of gas detectors and provides compliance information along with exposure trends and warning information.

### PRIOR ART DOCUMENTS

### Patent Document

Patent Document 1: Japanese Patent No. 5893376

### BRIEF DESCRIPTION OF THE DRAWINGS

FIG. 1 is a diagram illustrating an example of a system configuration of an information processing system.

FIG. 2 is an example of a functional block of a sensor.

FIG. 3 is a diagram illustrating an example of a functional block of an information processing apparatus.

FIG. 4 is a flowchart illustrating an example of a procedure for transmitting statistical information of the sensor.

FIG. 5 is a flowchart illustrating an example of a processing procedure in which the information processing apparatus generates an environment control instruction for an environment control apparatus.

FIG. 6 is a diagram illustrating an example of a hardware configuration.

### DESCRIPTION OF EXEMPLARY EMBODIMENTS

Hereinafter, embodiments of the present invention will be described. However, the following embodiments are not for limiting the invention according to the claims. In addition, not all of the combinations of features described in the embodiments are essential to the solution of the invention.

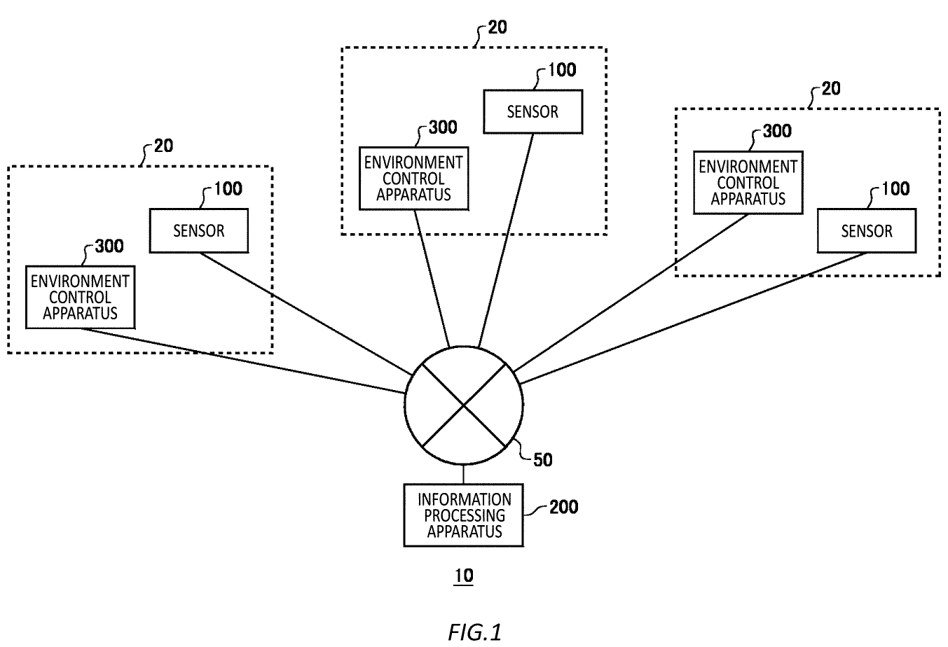

FIG. 1 is a diagram illustrating an example of a system configuration of an information processing system 10 according to the present embodiment. The information processing system 10 includes a plurality of sensors 100, an information processing apparatus 200, and an environment control apparatus 300. The sensor 100, the information processing apparatus 200, and the environment control apparatus 300 may be computers including a CPU and a memory, and the CPU may execute various functions by executing various programs stored in the memory. The environment control apparatus 300 may be, for example, a ventilation facility, or an air conditioning facility such as air conditioners, air cleaners, and humidifiers. The sensor 100, the information processing apparatus 200, and the environment control apparatus 300 communicate with each other via a network 50.

The plurality of sensors 100 may be provided at different positions in a target space 20. The target space 20 is, for example, an indoor area of a building such as a house, a store, or an office, and may be an indoor area of transportation equipment such as a car, a ship, a railway vehicle, or an airplane. In addition, the space 20 may be a plurality of different rooms, buildings, or locations. The sensor 100 measures environment information regarding the environment in the target space 20. The information processing apparatus 200 derives, based on the statistical information of the environment information, a comfort index which is an index representing a comfort level in the target space 20.

The environment information may represent at least one of a gas concentration, a dust amount, a temperature, a humidity, an atmospheric pressure, a noise, an illuminance, a vibration, an electromagnetic wave, a sound wave, an X-ray dose, a radiation dose, an ozone concentration, a flow velocity, an airflow, the number of living organisms, the postures of the living organisms, or the positions of the living organisms to be measured in the target space 20. The sensor 100 may have a battery, and may be driven by electrical power from the battery. Thereby, even when there is no power supply facility for supplying power to the sensor 100 in the target space 20, the sensor 100 can be driven. By driving the sensor 100 with the battery, the sensor can be installed in and moved to an arbitrary location. The sensor 100 may be mounted on a mobile body. The mobile body is a concept that includes a flying object moving in the air, a vehicle moving on the ground, a ship moving on water, or the like. The mobile body may be an unmanned mobile body. The flying object moving in the air is a concept that includes another aircraft, an airship, a helicopter, or the like which moves in the air in addition to an unmanned aerial vehicle (UAV). That is, the target space 20 may be different locations over time. The mobile body may be a living organism such as a human, livestock, an agricultural crop, or a plant.

When the sensor 100 is a gas sensor, the sensor may measure a gas concentration of gas to be measured, according to a non-dispersive infrared absorption method, a photoacoustic method, a solid electrolyte method, a thermal conduction method, an acoustic wave method, or a capacitance method. The gas to be measured may be carbon dioxide, a volatile organic compound (VOC), or oxygen. The gas to be measured may be a flammable gas such as methane, propane, ethanol, or hydrogen. The gas to be measured may be a refrigerant gas such as Freon, alternative Freon, R32, or R1234yf. The gas to be measured may be a toxic gas such as carbon monoxide, hydrogen sulfide, formaldehyde, or ammonia, and may be a hydrogen carrier gas such as methane or MCH (methylcyclohexane). The gas to be measured may be a greenhouse gas such as nitrous oxide. The sensor 100 may be a dust sensor, a temperature/humidity sensor, a barometric pressure sensor, a noise sensor, an illuminance sensor, a vibration sensor, a microphone, an electromagnetic wave measuring instrument, a millimeter wave radar, an X-ray measuring instrument, a radiation measuring instrument, a blood sugar level sensor, a blood pressure sensor, a heart rate sensor, a thermometer, or an airflow tester.

The sensor 100 is fixed at a predetermined position in the target space 20. The sensor 100 is fixed with a bolt or the like so that it cannot be easily detached from the predetermined position in the target space 20. The sensor 100 may have a tamper-resistant function, and when detached from the predetermined position, detect the detachment.

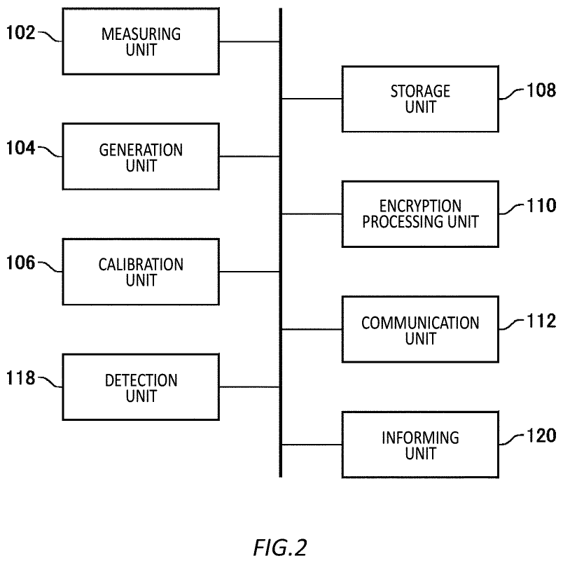

FIG. 2 is an example of a functional block of the sensor 100. The sensor 100 includes a measuring unit 102, a statistics generation unit 104, a calibration unit 106, a storage unit 108, an encryption processing unit 110, a communication unit 112, a detection unit 118, and an informing unit 120.

The measuring unit 102 measures environment information regarding the environment in the target space 20. The measuring unit 102 measures the environment information regarding the environment in the target space 20 to be authenticated at predetermined intervals, for example, at intervals of once every 60 seconds or more. The measuring unit 102 may include a light emitting unit which emits an infrared ray and a light receiving unit which receives the infrared ray having transmitted through a gas to be measured, and may measure a gas concentration of the gas by using an infrared absorption characteristic of the gas to be measured. The measuring unit 102 may measure gas information representing a concentration of gas in the target space 20, as the environment information. The measuring unit 102 may measure carbon dioxide information representing a concentration of carbon dioxide in the target space 20 as the gas information. In addition to the gas information, the measuring unit 102 may further measure temperature/humidity information representing a temperature and a humidity in the target space 20, as the environment information.

In addition, the measuring unit 102 may detect whether a measurement value of the measured environment information is an abnormal value. The measuring unit 102 may determines that a measurement value x is an abnormal value when an abnormality degree α(x) of a measurement value x, which is defined by following Expression 1 where an average value and a standard deviation in past measurement value data for a certain time are respectively denoted by μ and σ, is a predetermined threshold or more.

$$α ( x ) = \frac{{( x - μ )}^{2}}{{σ}^{2}} \qquad ( 1 )$$

In addition, the measuring unit 102 may set, as an abnormality degree β, the smallest distance, or one of the top distances when arranged in ascending order, among distances of the past measurement value data for a certain time and the measurement value x, and compare the same with a threshold, thereby determining that the measurement value x is an abnormal value. Here, the distance is a distance, which includes an absolute difference or a Mahalanobis distance, or the like, in a mathematical concept.

The statistics generation unit 104 generates, based on the environment information, statistical information regarding the environment of the space. The statistics generation unit 104 continuously generates the statistical information of the environment information measured by the measuring unit 102 over a predetermined period. The statistics generation unit 104 generates, for example, the statistical information including the average value, the minimum value, and the maximum value of the gas concentration in the predetermined period. The average value, the minimum value, and the maximum value of the gas concentration are examples of statistical values. The statistics generation unit 104 stores the statistical information in the storage unit 108. The statistics generation unit 104 may generate the statistical information including at least one of the average value, the maximum value, the minimum value, the variance, the moment, or the histogram of the gas concentration in a predetermined period. The statistical information may include an abnormal value of the environment information and the frequency of the abnormal value. When generating the statistical information, a machine learning model may be used. The statistics generation unit 104 may generate the statistical information based on the environment information of a plurality of sensors. In addition, the statistics generation unit 104 for generating the statistical information may generate macro statistical information obtained by further statistically processing the statistical information, the position information, and the sensor identification information of the plurality of sensors. The sensor 100 further includes a macro statistical information generation unit, and when there are a plurality of target spaces 20, the macro statistical information generation unit may further statistically process the statistical information linked to the plurality of target spaces 20, and generate the macro statistical information representing where the statistical information linked to a specific target space 20 is statistically positioned.

In addition, the statistics generation unit 104 may generate the statistical information based on the environment information and the position information or the sensor identification information, and the health information of a user associated with the sensor 100. The health information of the user associated with the sensor 100 may include at least one of health information, which is inputted from the user or other equipment, of the user carrying the sensor 100, body temperature information, blood pressure information, blood gas information, heart rate information, blood sugar information, cardiopulmonary function information, breathing sound information, brain wave information, or exercise information of the user or a user cohabitant. In addition, the health information which is inputted from the user or other equipment may include at least one of body weight, height, body fat percentage, muscle mass, bone density, disease status, symptoms, presence or absence of fatigue, diet information, depression, mood, exercise history, sleep information, drinking information, smoking information, drowsiness information, medication information, defecation information, or caffeine intake information. For example, a regional disease rate or a regional infection rate may be calculated from the disease status and the position information, and a health index may be calculated by using the environment information.

The communication unit 112 includes a wireless communication interface and wirelessly transmits the statistical information to the information processing apparatus 200. The communication unit 112 may transmit the statistical information to the information processing apparatus 200 by wire. The encryption processing unit 110 encrypts information that is transmitted by the communication unit 112.

The storage unit 108 stores sensor identification information for uniquely identifying the sensor 100. The sensor identification information may be a serial number of the sensor 100. The sensor identification information may represent at least one of an apparatus ID, a date of manufacture of an apparatus, a product model number, manufacturer/seller and their contact points, a manufacturing location, a warranty period, an operating principle of an apparatus, an apparatus specification, a recommended operating environment (temperature, humidity), a name of an apparatus, a manufacturing lot number, a safety standard that an apparatus meets, a disposal method, an applicable standard for a specific substance (JIS, JEDEC, etc.), a record regarding self-calibration (time, calibration data used, adjustment parameters before and after calibration), an identification number on a network, an installer name (store name, etc.), an installer contact point (telephone number, e-mail address, website URL, etc.), an installation location address, an installation date and time, or a record regarding a consent from a user to the effect that the environment information may be transmitted to an outside. The storage unit 108 may store the statistical information in association with the sensor identification information.

The communication unit 112 may transmit the sensor identification information along with the statistical information to the information processing apparatus 200. The information processing apparatus 200 may associate the statistical information with the sensor identification information and register the result in a storage unit such as a database which the information processing apparatus 200 can access. The communication unit 112 may transmit, to the information processing apparatus 200, the installer name, the installer contact point (telephone number, e-mail address, website URL), the installation location address, and the installation date and time, and the apparatus ID of the sensor 100 in response to an instruction from the user. The information processing apparatus 200 may associate installer information, regarding the installer, including the installer name, the installer contact point (telephone number, e-mail address, website URL), the installation location address, and the installation date and time with the apparatus ID of the sensor 100, and register the result in a database on a network which the information processing apparatus 200 can access. A casing of the sensor 100 may be provided with link information such as a bar code, a two-dimensional code, or RFID representing information enabling access to a website for associating the installer information and the apparatus ID. For example, by reading the link information with a camera mounted on a portable terminal such as a smartphone, the portable terminal accesses the website. Then, when the user inputs the installer information and the apparatus ID via the portable terminal, the installer information and the apparatus ID may be associated and registered in the database.

A level of access authority to the environment information stored in the storage unit 108 may be higher than a level of access authority to the statistical information stored in the storage unit 108. The environment information is detailed raw data regarding the target space 20, and has a higher level of confidentiality than that of the statistical information. Therefore, from a standpoint of privacy protection, access to the environment information stored in the storage unit 108 is preferably restricted to only a specific user such as an administrator of the target space 20. The statistics generation unit 104 may delete the environment information stored in the storage unit 108 in response to generating the statistical information.

The detection unit 118 detects whether the sensor 100 is installed at a predetermined position. The detection unit 118 has a tamper-resistant function, and, for example, when the detection unit detects that the screw fixing the sensor 100 is detached, the detection unit updates the installation information stored in the storage unit 108 and representing whether the sensor 100 is installed at a predetermined position. That is, when the sensor 100 is detached, the detection unit 118 updates the installation information into installation information representing that the sensor 100 is not installed at a predetermined position. The installation information may be flag information representing whether the sensor 100 is installed at a predetermined position, i.e., bit information representing “0” or “1”. The detection unit 118 may monitor a voltage of a pin connected to the casing of the sensor 100 by the tamper-resistant function, and when the voltage exceeds a threshold, the detection unit may set the flag by updating the bit information from “0” to “1”, for example. The voltage of the pin fluctuates and exceeds the threshold when the casing is opened, for example.

The detection unit 118 may include a position information acquisition function of acquiring position information representing a current position where the sensor 100 is present. The detection unit 118 may include a GNSS receiver, such as a Global Positioning System (GPS) receiver, a Global Navigation Satellite System (GLONASS) receiver, or a BeiDou Navigation Satellite System (BDS) receiver, as the position information acquisition function. The detection unit 118 may determine that the sensor 100 has been detached, and to update the installation information, when the current position of the sensor 100 is different from a position predetermined as an installation position. The communication unit 112 may transmit the installation information along with the statistical information to the information processing apparatus 200. Alternatively, the position information obtained from a position information acquisition function may be used to identify the position of the sensor 100 or the target space 20, and the position of the sensor 100 or the target space 20 may be stored in the storage unit 108.

The communication unit 112 may transmit failure information, which represents whether the sensor 100 is malfunctioning, to the information processing apparatus 200 along with the statistical information.

The calibration unit 106 performs calibration of the sensor 100. A characteristic of the sensor 100 may change along with the lapse of time. The characteristic of the sensor 100 refers to a characteristic of an optical element or the like, for example, when the sensor 100 is an optical element and is also a CO₂ (carbon dioxide) sensor according to the non-dispersive infrared absorption method in which a gas concentration is measured by infrared light. The characteristic of an optical element or the like may change over time. For this reason, the sensor 100 performs calibration for correcting measuring accuracy.

The calibration unit 106 may perform calibration based on a gas concentration calculated by itself and a predetermined reference gas concentration in the target space 20 to be measured. The calibration unit 106 may correct a coefficient for calculating the gas concentration so that the gas concentration calculated by itself matches the reference gas concentration when a condition under which the gas concentration in the target space 20 becomes the reference gas concentration is satisfied. The sensor 100 may correct the coefficient so that a minimum value of the gas concentration calculated by itself within a predetermined period matches the reference gas concentration. The condition under which the gas concentration in the target space 20 becomes the reference gas concentration is, for example, a condition that a time point at which the calibration unit 106 performs calibration is a time zone in which a possibility of presence of a living organism such as a human is low. The condition under which the gas concentration in the target space 20 becomes the reference gas concentration is, for example, a condition that a time point at which the calibration unit 106 performs calibration is within a predetermined period after predetermined ventilation of the target space 20 is performed for a predetermined period or longer.

The storage unit 108 may store calibration information including at least one of a calibration time at which calibration should be performed by the calibration unit 106, a date and time of calibration performed by the calibration unit 106, a calibration method, or calibrator information. The calibration method is a method of calibration that is performed by the calibration unit 106. The calibration method may represent, for example, a ventilation time of the target space 20, a ventilation method such as a ventilation setting of a ventilation apparatus, a time from ventilation to calibration execution, and the like. The calibration method may represent, for example, that calibration is performed within a predetermined time zone after ventilation of a predetermined ventilation time is performed in a specific time zone at night when no person is present. The communication unit 112 may transmit the calibration information along with the statistical information to the information processing apparatus 200. The calibration method may represent at least one of a type of gas to be measured, a concentration, a concentration score, a traceability system, a concentration accuracy, a gas component, a gas purchase date/calibration certificate issue date, a gas seller, a gas purchaser, a container symbol number, an expiration date of gas, a type of an adjustment parameter (for example, zero, span, offset, sensitivity), environment information at the time of calibration (temperature, humidity, atmospheric pressure, and date and time), or a residual pressure of calibration gas.

The informing unit 120 may inform an outside of a message representing the calibration time stored in the storage unit 108. The informing unit 120 may display a message representing the calibration time on a display unit of the sensor 100. The informing unit 120 may transmit a message representing the calibration time to the administrator of the target space 20 via the communication unit 112 by an e-mail or the like. The informing unit 120 may deliver a message representing the calibration time to the administrator of the target space 20 in writing.

The storage unit 108 may store associated information including at least one of a spatial volume of the target space 20, a designed ventilation amount of the target space 20, operator information for identifying an operator who uses the target space 20, an installation time of the sensor 100, or an installation location of the sensor 100. The communication unit 112 may transmit the associated information along with the statistical information to the information processing apparatus 200. The associated information may include information regarding the environment control apparatus 300 installed in the target space 20. The information regarding the environment control apparatus 300 may include identification information for uniquely identifying the environment control apparatus 300.

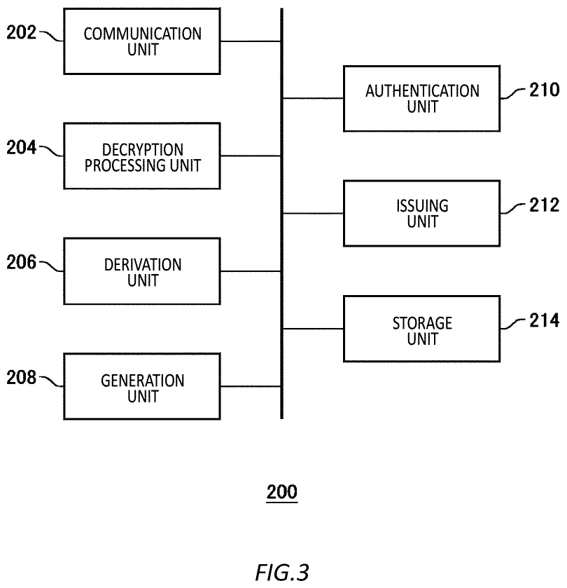

FIG. 3 illustrates an example of a functional block of the information processing apparatus 200. The information processing apparatus 200 includes a communication unit 202, a decryption processing unit 204, a derivation unit 206, a generation unit 208, an authentication unit 210, an issuing unit 212, and a storage unit 214.

The communication unit 202 receives the statistical information of the environment information from each of the plurality of sensors 100. The communication unit 202 may receive the sensor identification information, the installation information, and the associated information along with the statistical information from the sensor 100. The decryption processing unit 204 decrypts the encrypted information. The decryption processing unit 204 may decrypt the encrypted statistical information, sensor identification information, installation information, calibration information, and associated information according to a predetermined decryption algorithm.

The derivation unit 206 derives, based on the statistical information of the environment information transmitted from the sensor 100, the comfort index which represents the comfort level in the target space 20. The derivation unit 206 may derive the comfort index of the target space 20 based on a plurality of pieces of statistical information of the environment information respectively transmitted from the plurality of sensors 100 provided in the target space 20.

The derivation unit 206 may derive, according to a predetermined derivation method and based on the statistical information, a health index representing, as the comfort level of the target space, whether it is possible to spend time healthily in the target space. When the statistical information is the concentration of carbon dioxide, the derivation unit 206 may derive, as the health index, the inverse (1/P) of an infection probability P derived based on the following expression according to the Wills-Rilely model. The target space may be, for example, an indoor room.

$$P = 1 - exp ( - n ) \\ n = ( N - 1 ) ( 1 - {η}_{i m} ) {η}_{I} ( 1 - {m}_{i n} ) ( 1 - {m}_{ex} ) {E}_{p} \frac{C - {C}_{0}}{{E}_{p C 02}} ( \frac{{λ}_{0}}{λ} ) BD$$

Here,
n is a minimum infection unit (quanta),
N is the number of occupants (people),
η₁ is a regional infection rate,
mₑₓ is a mask exhalation inhibition coefficient,
mᵢₙ is a mask inhalation inhibition coefficient,
ηᵢₘ is a resistance rate,
Eₚ is a quanta emission rate (quanta/h),
E_{pCO2} is a CO₂ emission amount (m³/h·people),
λ₀ is the number of times of ventilation (/h),
λ is an infectivity attenuation rate (/h),
D is contact time (h),
C is a CO₂ concentration of the room,
C₀ is a CO₂ concentration of the outside air, and
B is a breathing amount (m³/h).

The sensor 100 measures the CO₂ concentration C of the room as the environment information. Each parameter other than the CO₂ concentration C of the room may be stored in advance in the storage unit 108 for the sensor 100 or each target space 20 provided with sensor 100. Alternatively, the derivation unit 206 may acquire each parameter via the network 50 from a server which manages each parameter.

According to the predetermined derivation method, the derivation unit 206 may derive, as the comfort information, a work efficiency representing, as the comfort level of the target space, whether work such as learning or work can be performed with concentration in the target space. When the statistical information is a temperature in the target space, the derivation unit 206 may derive, based on the following expression, work efficiency RPₐₗₗ by summing work efficiency RP_{V} for a ventilation amount V (cubic meters/minute) and work efficiency RP_{T} for a temperature T (° C.).

$${RP}_{all} = {RP}_{V} + {RP}_{T} \\ {RP}_{V} = exp ( ( - 161.822 {V}^{- 1} - 0.368 V Ln ( 0.472 V ) + 1.827 V - {y}_{0} ) / 1000 ) \\ {y}_{0} = - 161.822 {X}_{R}^{- 1} - 0.368 {X}_{R}^{- 1} Ln ( 0.472 {X}_{R}^{- 1} ) + 1.827 {X}_{R}^{- 1} \\ {RP}_{T} = - 0.4685328 + 0.1647524 T - 0.0058274 {T}^{2} + 0.0000623 {T}^{3}$$

where X_{R} is 0.5 (cubic meters/minute) (17.66 (cubic feet/minute)).

Ln is a natural logarithm. The sensor 100 measures the temperature of the room as the environment information. Ln may be stored in advance in the storage unit 108.

When the statistical information is a temperature and a humidity, the derivation unit 206 may derive a heat index as the health index. For example, the heat index may be a wet bulb globe temperature (WBGT) value.

When the statistical information is sound, the posture or vital signs of a person in the target space, the derivation unit 206 may derive, as the health index, the biological state index of the person in the target space. For example, the biological state index may be an index representing a degree of health that takes into account states such as presence or absence of sleep apnea syndrome, presence or absence of falls, or wake-up or sleep.

When the statistical information is the number or postures of living organisms, the derivation unit 206 may derive, as the health index, an index representing the possibility that a person is left behind. For example, in a case where the target space is an indoor area of a car, and the number of living organisms is detected by a millimeter wave radar, when the millimeter wave radar detects the presence of a living organism even though the door of the car is locked, the derivation unit 206 may derive an index indicating that there is a high possibility that a person is left behind.

The generation unit 208 generates the comfort information representing the comfort index of the target space 20. The generation unit 208 may generate, as the comfort information, image information representing the comfort index to be displayed on a display unit of a user terminal such as a tablet terminal or a wearable terminal.

The authentication unit 210 authenticates that the statistical information is information regarding the target space 20. The authentication unit 210 may authenticate, based on the statistical information and the sensor identification information, that the statistical information is information regarding the target space 20. The authentication unit 210 may authenticate, based further on the installation information, that the statistical information is information regarding the target space 20. Note that the information processing apparatus 200 may not be provided with the authentication unit 210.

When the statistical information has been authenticated by the authentication unit 210, the issuing unit 212 issues an electronic certificate for the comfort information. The electronic certificate certifies that the comfort index represented by the comfort information is an index derived based on highly reliable statistical information without tampering for the target space 20. In addition, the electronic certificate certifies that the comfort index represented by the comfort information represents the degree of comfort of the target space 20.

The authentication unit 210 may authenticate the statistical information based on at least one of the sensor identification information, the installation information, the calibration information, or the associated information in addition to the statistical information. The authentication unit 210 may authenticate the statistical information based on the statistical information, the sensor identification information, the installation information, the calibration information, and the associated information. The storage unit 214 may store an authentication condition in association with the sensor identification information or the associated information. The authentication unit 210 may authenticate the statistical information when at least one of the statistical information, the installation information, or the calibration information satisfies the authentication condition.

The authentication unit 210 refers to the installation information and determines whether the sensor 100 is installed at a predetermined position. The authentication unit 210 refers to the calibration information and determines, based on a predetermined calibration method, whether the calibration of the sensor 100 has been performed in a predetermined period by an authorized calibrator. The authentication unit 210 authenticates the statistical information when the authentication condition for each information is satisfied, and the statistical information satisfies the authentication condition. When there are a plurality of sensors 100, the authentication unit 210 may further statistically process and authenticate the statistical information sent from the plurality of sensors 100.

When the authentication unit 210 successfully authenticates the statistical information, the issuing unit 212 issues an electronic certificate for the comfort information based on the statistical information. The electronic certificate is an electronic document to which an electronic signature of an approval agency has been applied, the electronic document showing that the comfort index represented by the comfort information is the index based on the authenticated statistical information. In addition, the electronic certificate certifies that the comfort index represented by the comfort information represents the degree of comfort of the target space 20. The communication unit 202 transmits the electronic certificate to a predetermined destination such as the administrator of the target space 20. The issuing unit 212 may associate the electronic authentication certificate with the installer information and the apparatus ID via the communication unit 202 and register the result in the database accessible by a third party. Thereby, by accessing the database using a search system or the like, a third party can easily check whether the statistical information has been authenticated, that is, whether the comfort index based on the statistical information is accurate information which satisfies the predetermined reference and is free from irregularities. The authentication unit 210 may be provided in a personal computer, a server, a smartphone, a tablet terminal, or a wearable terminal, or the like.

The generation unit 208 may generate an environment control instruction for controlling the environment control apparatus 300 which causes changes in the environmental state of the target space 20 based on the comfort information. When the comfort index is smaller than a threshold, the generation unit 208 may generate the environment control instruction for controlling the environment control apparatus 300 to cause changes in the environmental state of the target space such that the comfort index becomes equal to or greater than the threshold.

The communication unit 202 communicates with the environment control apparatus 300. The communication unit 202 transmits the environment control instruction to the environment control apparatus 300. The environment control apparatus 300 changes the environmental state of the target space 20 in response to the environment control instruction.

For example, when the health index is smaller than a threshold, the generation unit 208 may generate, as the environment control instruction, each control instruction for a ventilation fan or an air conditioner such that the airflow rate of the ventilation fan is increased to increase the ventilation amount and such that the driving of the air conditioner is controlled so that the temperature of the target space 20 falls within a predetermined temperature range, and the humidity of the target space 20 falls within a predetermined humidity range, in order that the health index becomes equal to or greater than the threshold. The communication unit 202 may transmit each control instruction to each of the ventilation fan and the air conditioner. The storage unit 214 may store, for each target space 20, the airflow rate of the ventilation fan, the temperature range, and the humidity range for maintaining the health index at or above the threshold.

For example, when the work efficiency is smaller than a threshold, the generation unit 208 may generate, as the environment control instruction, each control instruction for the ventilation fan and the air conditioner such that the airflow rate of the ventilation fan is increased to increase the ventilation amount and such that the driving of the air conditioner is controlled so that the temperature of the target space 20 falls within the predetermined temperature range, and the humidity of the target space 20 falls within the predetermined humidity range, in order that the work efficiency becomes equal to or greater than the threshold. The communication unit 202 may transmit each control instruction to each of the ventilation fan and the air conditioner.

For example, the health index and the work efficiency can be increased by performing P-control or PID-control on the ventilation fan and the air conditioner according to the following expression for the temperature T and the ventilation amount V.

$$T \to T + α \frac{\partial S}{\partial T} \\ V \to V + α \frac{\partial S}{\partial V}$$

Here, α is a constant with a sufficiently small absolute value (for example, α=0.5), is a positive value when improvement occurs as the health index or the work efficiency increases, and is a negative value when improvement occurs as the health index or the work efficiency decreases.

A cost-based term T_{c} may be added to the health index or the work efficiency. For example, the cost term T_{c} may be defined by T_{c}=1−aexp(bc(T, V)). Here, a and b are positive constants, and c is a monotonically increasing function of each of the temperature T and the ventilation amount V. Thereby, the generation unit 208 can generate the environment control instruction for controlling the ventilation fan or the air conditioner in order to adjust the temperature or the ventilation amount in consideration with cost. For example, it is possible to prevent an infinite divergence in the ventilation amount.

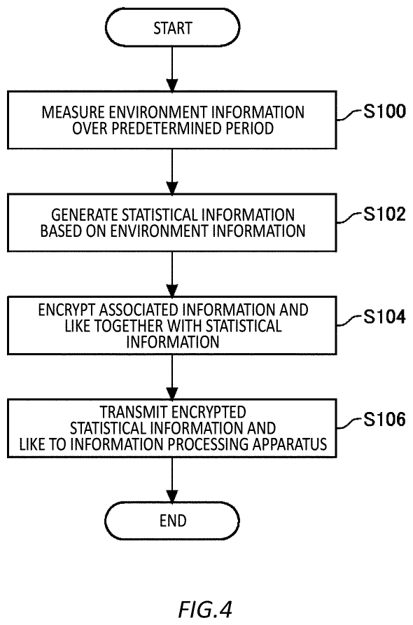

FIG. 4 is a flowchart illustrating an example of a procedure for transmitting the statistical information of the sensor 100.

The measuring unit 102 measures environment information such as a gas concentration to be measured in the target space 20 at predetermined intervals (for example, once every 60 seconds) over a predetermined period (for example, one month or the like) (S100). The measuring unit 102 may store the environment information in the storage unit 214. The statistics generation unit 104 generates statistical information based on the environment information measured in the predetermined period and stored in the storage unit 108 (S102). When the generation of the statistical information is completed, the statistics generation unit 104 may delete the environment information stored in the storage unit 108. The encryption processing unit 110 acquires the sensor identification information, the installation information, the calibration information, and the associated information from the storage unit 108 and encrypts the information together with the statistical information (S104). The communication unit 112 transmits the encrypted statistical information and the like to the information processing apparatus 200 (S106).

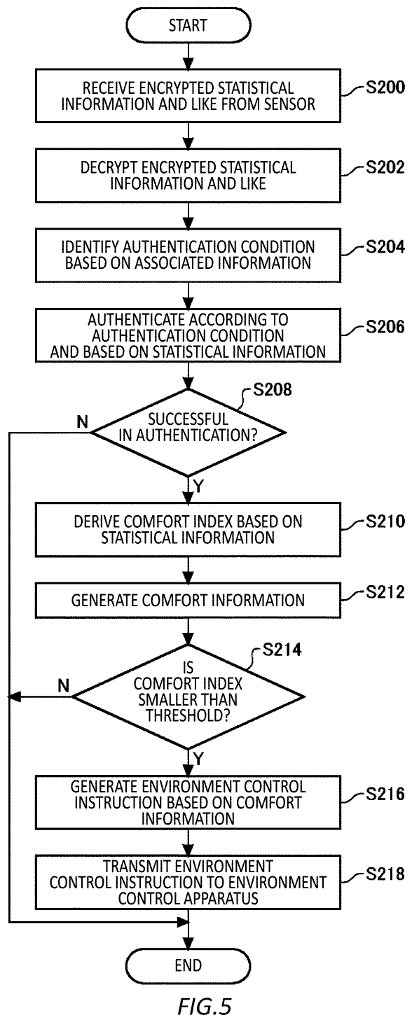

FIG. 5 is a flowchart illustrating an example of a processing procedure in which the information processing apparatus 200 generates the environment control instruction for the environment control apparatus 300.

The communication unit 202 receives the encrypted statistical information and the like from the sensor 100 (S200). The decryption processing unit 204 decrypts the encrypted statistical information and the like (S202). The authentication unit 210 refers to the authentication conditions stored in the storage unit 214 and identifies, based on the decrypted associated information, an authentication condition associated with the target space 20 (S204).

The authentication unit 210 performs authentication according to the authentication condition, based on the statistical information (S206). The authentication unit 210 determines whether authentication has been successful (S208). If the authentication has been successful, the derivation unit 206 derives, based on the statistical information, the comfort index representing the comfort level of the target space 20 (210). The generation unit 208 generates the comfort information representing the comfort index of the target space 20 (S212). The generation unit 208 may generate, as the comfort information, image information representing the comfort index to be displayed on a display unit of a user terminal such as a tablet terminal.

Furthermore, the generation unit 208 determines whether the comfort index is smaller than the threshold (S214). When the comfort index is smaller than the threshold, the generation unit 208 generates the environment control instruction for controlling the environment control apparatus 300 which causes changes in the environmental state of the target space 20 based on the comfort information (S216). When the comfort index is smaller than a threshold, the generation unit 208 may generate the environment control instruction for controlling the environment control apparatus 300 to cause changes in the environmental state of the target space such that the comfort index becomes equal to or greater than the threshold. The communication unit 202 transmits the environment control instruction to the environment control apparatus 300 (S218).

Hereinbefore, according to the present embodiment, since the plurality of sensors 100 do not transmit, to the information processing apparatus 200, the environment information as it is, but generate the statistical information from the environment information and transmits the statistical information to the information processing apparatus 200, the processing load on the information processing apparatus 200 which aggregates information from the plurality of sensors 100 can be reduced.

Furthermore, since the environment control apparatus 300 can be controlled by the environment control instruction corresponding to the comfort index based on the statistical information, the comfort level in the target space 20 can be more reliably secured.

The sensor 100 may be provided with a reception unit. The reception unit may receive the electronic certificate certifying that the comfort index or the comfort information represents the comfort level of the target space. In addition, the reception unit may receive the macro statistical information obtained by further statistically processing the statistical information of the plurality of sensors 100. For example, when there are a plurality of target spaces 20, the statistical information linked to the plurality of target spaces 20 may be further statistically processed, and the macro statistical information may represent where the statistical information linked to a specific target space 20 is statistically positioned. Specifically, the macro statistical information may be a ranking or histogram of the statistical information of the plurality of target spaces 20. In addition, the reception unit may receive the statistical information linked to the target space 20 or evaluation information based on the macro statistical information, and the evaluation information may be categorized in five levels: not acceptable, acceptable, good, excellent, and outstanding. Furthermore, the reception unit may receive improvement information or reward information based on the evaluation information linked to the target space 20. For example, the improvement information may be an improvement plan or a countermeasure plan for improving the evaluation information of the target space 20 when the evaluation information linked to the target space 20 is poor, or

when the statistical information linked to the target space 20 satisfies a certain numerical condition. In addition, for example, the reward information may be information representing praise or may be reward points when the evaluation information linked to the target space 20 is good, when the statistical information linked to the target space 20 satisfies a certain numerical condition, or when a person who has viewed the statistical information linked to the target space 20 has performed evaluation. In addition, the reception unit may not be in the sensor 100, but may be in a smartphone, a tablet, or a PC. In addition, the information received by the reception unit may be displayed on a display unit. The display unit may be in the sensor 100, or may be in a smartphone, a tablet, a PC, or a wearable terminal.

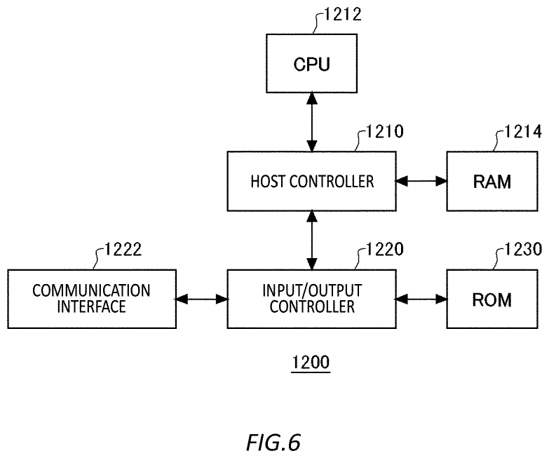

FIG. 6 illustrates an example of a computer 1200 in which a plurality of aspects of the present invention may be embodied in whole or in part. Programs installed in the computer 1200 can cause the computer 1200 to function as operations associated with the apparatus according to the embodiments of the present invention or one or more “units” of the apparatuses. Alternatively, the programs can cause the computer 1200 to execute the operations or the one or more “units”. The programs can cause the computer 1200 to execute a process according to the embodiments of the present invention or steps of the process. Such programs may be executed by a CPU 1212 to cause the computer 1200 to perform specific operations associated with some or all of the blocks in the flowcharts and block diagrams described in the present specification.

The computer 1200 according to the present embodiment includes the CPU 1212 and a RAM 1214, which are mutually connected by a host controller 1210. The computer 1200 also includes a communication interface 1222 and an input/output unit, which are connected to the host controller 1210 via an input/output controller 1220. The computer 1200 also includes a ROM 1230. The CPU 1212 operates according to the programs stored in the ROM 1230 and the RAM 1214, thereby controlling each unit.

The communication interface 1222 communicates with other electronic devices via a network. A hard disk drive may store the programs and data used by the CPU 1212 in the computer 1200. The ROM 1230 stores therein boot programs or the like executed by the computer 1200 at the time of activation, and/or stores programs depending on hardware of the computer 1200. A program is provided via a computer-readable recording medium such as a CD-ROM, a USB memory, or an IC card, or via a network. The programs are installed in the RAM 1214 or the ROM 1230 which is also an example of the computer readable recording medium, and executed by the CPU 1212. Information processing written in these programs is read by the computer 1200, and provides cooperation between the programs and the various types of hardware resources described above. An apparatus or a method may be configured by implementing operations or processing of information according to a use of the computer 1200.

For example, in a case where a communication is performed between the computer 1200 and an external device, the CPU 1212 may execute a communication program loaded in the RAM 1214 and instruct the communication interface 1222 to perform communication processing based on a process written in the communication program. The communication interface 1222, under the control of the CPU 1212, reads transmission data stored in a transmission buffer region provided in a recording medium such as the RAM 1214 or the USB memory, transmits the read transmission data to the network, or writes reception data received from the network to a reception buffer region or the like provided on the recording medium.

In addition, the CPU 1212 may cause all or necessary portion of a file or a database stored in an external recording medium such as a USB memory, to be read by the RAM 1214, and execute various types of processing on the data on the RAM 1214. Then, the CPU 1212 may write the processed data back in the external recording medium.

Various types of information, such as various types of programs, data, tables, and databases, may be stored in the recording medium to undergo information processing. The CPU 1212 may execute, on the data read from the RAM 1214, various types of processing including various types of operations, information processing, conditional judgement, conditional branching, unconditional branching, information retrieval/replacement, or the like described throughout the present disclosure and specified by instruction sequences of the programs, to write the results back to the RAM 1214. In addition, the CPU 1212 may retrieve information in a file, a database, or the like in the recording medium. For example, when a plurality of entries each having an attribute value of a first attribute associated with an attribute value of a second attribute are stored in the recording medium, the CPU 1212 may retrieve, out of the plurality of entries, an entry with the attribute value of the first attribute specified that meets a condition, read the attribute value of the second attribute stored in the entry, and thereby acquire the attribute value of the second attribute associated with the first attribute meeting a predetermined condition.

The programs or software module described above may be stored on the computer 1200 or in a computer readable storage medium near the computer 1200. In addition, a recording medium such as a hard disk or a RAM provided in a server system connected to a dedicated communication network or the Internet can be used as the computer readable storage medium, so that the programs are provided to the computer 1200 via the network.

A computer readable medium may include any tangible device that can store instructions to be executed by a suitable device. As a result, the computer readable medium having instructions stored therein includes an article of manufacture including instructions which can be executed to create means for performing operations specified in the flowcharts or block diagrams. Examples of the computer readable medium may include an electronic storage medium, a magnetic storage medium, an optical storage medium, an electromagnetic storage medium, a semiconductor storage medium, and the like. More specific examples of the computer readable medium may include a floppy (registered trademark) disk, a diskette, a hard disk, a random access memory (RAM), a read-only memory (ROM), an erasable programmable read-only memory (EPROM or flash memory), an electrically erasable programmable read-only memory (EEPROM (registered trademark)), a static random access memory (SRAM), a compact disc read-only memory (CD-ROM), a digital versatile disk (DVD), a Blu-ray (registered trademark) disk, a memory stick, an integrated circuit card, and the like.

Computer readable instructions may include either a source code or an object code written in any combination of one or more programming languages. The source code or the object code includes a conventional procedural programming language. The conventional procedural programming language may be assembler instructions, instruction-set-architecture (ISA) instructions, machine instructions, machine dependent instructions, microcode, firmware instructions, state-setting data, or an object oriented programming language such as Smalltalk (registered trademark), JAVA (registered trademark), C++, etc., and programming languages, such as the “C” programming language or similar programming languages. Computer readable instructions may be provided to a processor of a general purpose computer, special purpose computer, or other programmable data processing device, or to programmable circuitry, locally or via a local area network (LAN), a wide area network (WAN) such as the Internet, etc. The processor or the programmable circuitry may execute the computer readable instructions in order to create means for performing operations specified in the flowcharts or block diagrams. Examples of the processor include a computer processor, a processing unit, a microprocessor, a digital signal processor, a controller, a microcontroller, and the like.

While the present invention has been described by way of the embodiments, the technical scope of the present invention is not limited to the above described embodiments. It is apparent to persons skilled in the art that various alterations or improvements can be made to the above described embodiments. It is also apparent from description of the claims that the embodiments to which such alterations or improvements are made can be included in the technical scope of the present invention.

It should be noted that the operations, procedures, steps, stages, and the like of each process performed by an apparatus, system, program, and method shown in the claims, embodiments, or diagrams can be realized in any order as long as the order is not indicated by “prior to,” “before,” or the like and as long as the output from a previous process is not used in a later process. Even if the operation flow is described using phrases such as “first” or “next” for the sake of convenience in the claims, specification, or drawings, it does not necessarily mean that the process must be performed in this order.

### (Item 1)

An information processing apparatus including:
a communication unit which receives statistical information transmitted from a sensor, the sensor having a measuring unit which measures environment information regarding environment in a target space and a statistics generation unit which generates, based on the environment information, the statistical information regarding the environment of the target space;
a derivation unit which derives, based on the statistical information, a comfort index representing a comfort level of the target space according to a predetermined derivation method; and
a generation unit which generates comfort information representing the comfort index of the target space.

### (Item 2)

The information processing apparatus according to item 1, wherein
the sensor includes a plurality of sensors, each being identical to the sensor and provided in the target space, and the communication unit receives the statistical information from each of the plurality of sensors, and
the derivation unit derives, based on the statistical information from each of the plurality of sensors, the comfort index according to the predetermined derivation method.

### (Item 3)

The information processing apparatus according to item 1, wherein the environment information represents at least one of a gas concentration, a dust amount, a temperature, a humidity, an atmospheric pressure, a noise, an illuminance, a vibration, an electromagnetic wave, a sound wave, an X-ray dose, a radiation dose, a flow velocity, an airflow, an ozone concentration, the number of living organisms, postures of the living organisms, or positions of the living organisms to be measured in the target space.

### (Item 4)

The information processing apparatus according to item 2, wherein the environment information includes carbon dioxide information representing a concentration of carbon dioxide of the target space.

### (Item 5)

The information processing apparatus according to item 4, wherein the environment information further includes temperature/humidity information representing a temperature and a humidity of the target space.

### (Item 6)

The information processing apparatus according to item 1, wherein the statistical information includes at least one of an average value, a maximum value, a minimum value, a variance, a moment, or a histogram of measured values which are the environment information.

### (Item 7)

The information processing apparatus according to item 1, wherein
the communication unit receives, from the sensor and along with the statistical information, sensor identification information that is for uniquely identifying the sensor, and
the information processing apparatus further includes:
the authentication unit authenticates, based on the statistical information and the sensor identification information, that the statistical information is information regarding the target space.

### (Item 8)

The information processing apparatus according to item 7, further including an issuing unit which, when the statistical information has been authenticated by the authentication unit, issues an electronic certificate certifying that the comfort information represents the comfort level of the target space.

### (Item 9)

The information processing apparatus according to item 7, wherein
the sensor is installed at a predetermined position of the target space,
the sensor has a detection unit which detects whether the sensor is installed at the predetermined position, and a storage unit which stores installation information representing whether the sensor is installed at the predetermined position,
the communication unit receives the installation information along with the statistical information from the sensor, and
the authentication unit authenticates, based further on the installation information, that the statistical information is information regarding the target space.

### (Item 10)

The information processing apparatus according to item 1, wherein
the derivation unit derives, according to the predetermined derivation method, a health index representing, as the comfort level of the target space, whether it is possible to spend time healthily in the target space, and
the generation unit generates, as the comfort information, health information representing the health index of the target space.

### (Item 11)

The information processing apparatus according to item 10, wherein
when a minimum infection unit (quanta) is denoted by n, a number of occupants (people) is denoted by N, a regional infection rate is denoted by η₁, a mask exhalation inhibition coefficient is denoted by mₑₓ, a mask inhalation inhibition coefficient is denoted by mᵢₙ, a resistance rate is denoted by ηᵢₘ, a quanta emission rate (quanta/h) is denoted by Eₚ, a CO₂ emission amount (m³/h·people) is denoted by E_{pCO2}, a number of times of ventilation (/h) is denoted by λ₀, an infectivity attenuation rate (/h) is denoted by λ, contact time (h) is denoted by D, a CO₂ concentration of a room is denoted by C, a CO₂ concentration of outside air is denoted by C₀, and a breathing amount (m³/h) is denoted by B, the derivation unit derives an infection probability P by using

$$P = 1 - exp ( - n ) \\ n = ( N - 1 ) ( 1 - {η}_{i m} ) {η}_{I} ( 1 - {m}_{i n} ) ( 1 - {m}_{ex} ) {E}_{p} \frac{C - {C}_{0}}{{E}_{p C 02}} ( \frac{{λ}_{0}}{λ} ) BD ,$$

and
derives an inverse (1/P) of the infection probability P as the health index.

### (Item 12)

The information processing apparatus according to item 10, wherein the statistics generation unit generates the statistical information based on the environment information and position information of the sensor or sensor identification information for uniquely identifying the sensor, and the health information.

### (Item 13)

The information processing apparatus according to item 1, wherein the derivation unit derives, according to the predetermined derivation method, a work efficiency representing, as the comfort level of the target space, whether it is possible to perform work with concentration in the target space.

### (Item 14)

The information processing apparatus according to item 13, wherein
When a work efficiency for a ventilation amount V (cubic meters/minute) is denoted by RP_{V}, a work efficiency for a temperature (° C.) is denoted by RP_{T}, X_{R} is 0.5 (cubic meters/minute), and Ln is a log, the derivation unit derives a work efficiency RPₐₗₗ of the target space by summing the work efficiency RP_{V} and the work efficiency RP_{T} using

$${RP}_{all} = {RP}_{V} + {RP}_{T} \\ {RP}_{V} = exp ( ( - 161.822 {V}^{- 1} - 0.368 V Ln ( 0.472 V ) + 1.827 V - {y}_{0} ) / 1000 ) \\ {y}_{0} = - 161.822 {X}_{R}^{- 1} - 0.368 {X}_{R}^{- 1} Ln ( 0.472 {X}_{R}^{- 1} ) + 1.827 {X}_{R}^{- 1} \\ {RP}_{T} = - 0.4685328 + 0.1647524 T - 0.0058274 {T}^{2} + 0.0000623 {T}^{3}$$

### (Item 15)

The information processing apparatus according to item 1, wherein
the communication unit communicates with an environment control apparatus which causes changes in an environmental state of the target space,
the generation unit generates an environment control instruction for controlling the environment control apparatus which causes the changes in the environmental state of the target space, based on the comfort information, and
the communication unit transmits the environment control instruction to the environment control apparatus.

### (Item 16)

The information processing apparatus according to item 15, wherein when the comfort index is smaller than a threshold, the generation unit generates the environment control instruction for the environment control apparatus to cause the changes in the environmental state of the target space such that the comfort index becomes equal to or greater than the threshold.

### (Item 17)

An information processing system including:
the information processing apparatus according to any one of items 1 to 16; and
at least one of sensors, each being identical to the sensor.

### (Item 18)

An information processing method including:
receiving statistical information from a sensor, the sensor having a measuring unit which measures environment information regarding environment in a target space and a generation unit which generates, based on the environment information, the statistical information regarding the environment of the target space;
deriving, based on the statistical information, a comfort index representing a comfort level of the target space according to a predetermined derivation method; and
generating comfort information representing the comfort index of the target space.

### (Item 19)

A program that causes a computer to function as:
a communication unit which receives statistical information from a sensor, the sensor having a measuring unit which measures environment information regarding environment in a target space and a generation unit which generates, based on the environment information, the statistical information regarding the environment of the target space;
a derivation unit which derives, based on the statistical information, a comfort index representing a comfort level of the target space according to a predetermined derivation method; and
a generation unit which generates comfort information representing the comfort index of the target space.

### EXPLANATION OF REFERENCES

10: information processing system;
20: target space;
50: network;
100: sensor;
102: measuring unit;
104: statistics generation unit;
106: calibration unit;
108: storage unit;
110: encryption processing unit;
112: communication unit;
118: detection unit;
120: informing unit;
200: information processing apparatus;
202: communication unit;
204: decryption processing unit;
206: derivation unit;
208: generation unit;
210: authentication unit;
212: issuing unit;
214: storage unit;
300: environment control apparatus;
1200: computer;
1210: host controller;
1212: CPU;
1214: RAM;
1220: input/output controller;
1222: communication interface;
1230: ROM.

What is claimed is:

1. An information processing apparatus comprising:
a communication unit which receives statistical information transmitted from a sensor, the sensor having a measuring unit which measures environment information regarding environment in a target space and a statistics generation unit which generates, based on the environment information, the statistical information regarding the environment of the target space;
a derivation unit which derives, based on the statistical information, a comfort index representing a comfort level of the target space according to a predetermined derivation method;
a generation unit which generates comfort information representing the comfort index of the target space; and
an authentication unit which authenticates, based on the statistical information, that the statistical information is information regarding the target space.

2. The information processing apparatus according to claim 1, wherein
the sensor includes a plurality of sensors, each being identical to the sensor and provided in the target space, and the communication unit receives the statistical information from each of the plurality of sensors, and
the derivation unit derives, based on the statistical information from each of the plurality of sensors, the comfort index according to the predetermined derivation method.

3. The information processing apparatus according to claim 1, wherein the environment information represents at least one of a gas concentration, a dust amount, a temperature, a humidity, an atmospheric pressure, a noise, an illuminance, a vibration, an electromagnetic wave, a sound wave, an X-ray dose, a radiation dose, a flow velocity, an airflow, an ozone concentration, or a number, postures, or positions of living organisms to be measured in the target space.

4. The information processing apparatus according to claim 2, wherein the environment information includes carbon dioxide information representing a concentration of carbon dioxide of the target space.

5. The information processing apparatus according to claim 4, wherein the environment information further includes temperature/humidity information representing a temperature and a humidity of the target space.

6. The information processing apparatus according to claim 1, wherein the statistical information includes at least one of an average value, a maximum value, a minimum value, a variance, a moment or a histogram of measured values which are the environment information.

7. The information processing apparatus according to claim 1, wherein
the communication unit receives, from the sensor and along with the statistical information, sensor identification information that is for uniquely identifying the sensor, and
the authentication unit authenticates, based on the statistical information and the sensor identification information, that the statistical information is information regarding the target space.

8. The information processing apparatus according to claim 7, further comprising an issuing unit which, when the statistical information has been authenticated by the authentication unit, issues an electronic certificate certifying that the comfort information represents the comfort level of the target space.

9. The information processing apparatus according to claim 7, wherein
the sensor is installed at a predetermined position of the target space,
the sensor has a detection unit which detects whether the sensor is installed at the predetermined position, and a storage unit which stores installation information representing whether the sensor is installed at the predetermined position,
the communication unit receives the installation information along with the statistical information from the sensor, and
the authentication unit authenticates, based further on the installation information, that the statistical information is information regarding the target space.

10. The information processing apparatus according to claim 1, wherein
the derivation unit derives, according to the predetermined derivation method, a health index representing, as the comfort level of the target space, whether it is possible to spend time healthily in the target space, and
the generation unit generates, as the comfort information, health information representing the health index of the target space.

11. The information processing apparatus according to claim 10, wherein
when a minimum infection unit (quanta) is denoted by n, a number of occupants (people) is denoted by N, a regional infection rate is denoted by η₁, a mask exhalation inhibition coefficient is denoted by mₑₓ, a mask inhalation inhibition coefficient is denoted by mᵢₙ, a resistance rate is denoted by ηᵢₘ, a quanta emission rate (quanta/h) is denoted by Eₚ, a CO₂ emission amount (m³/h·people) is denoted by E_{pCO2}, a number of times of ventilation (/h) is denoted by λ₀, an infectivity attenuation rate (/h) is denoted by λ, contact time (h) is denoted by D, a CO₂ concentration of a room is denoted by C, a CO₂ concentration of outside air is denoted by C₀, and a breathing amount (m³/h) is denoted by B, the derivation unit derives an infection probability P by using, [symbol]
, and
derives an inverse (1/P) of the infection probability P as the health index.

12. The information processing apparatus according to claim 1, wherein the statistics generation unit generates the statistical information based on the environment information and position information of the sensor or sensor identification information for uniquely identifying the sensor, and on health information of a user associated with the sensor, and
the information processing apparatus further comprises:
a macro statistical information generation unit which further statistically processes the statistical information linked to plurality of target spaces, each being identical to the target space, and generates macro statistical information representing where the statistical information linked to a specific target space is statistically positioned.

13. The information processing apparatus according to claim 1, wherein the derivation unit derives, according to the predetermined derivation method, a work efficiency representing, as the comfort level of the target space, whether it is possible to perform work with concentration in the target space.

14. The information processing apparatus according to claim 13, wherein
When a work efficiency for a ventilation amount V (cubic meters/minute) is denoted by RP_{V}, a work efficiency for a temperature (° C.) is denoted by RP_{T}, X_{R} is 0.5 (cubic meters/minute), and Ln is a natural logarithm, the derivation unit derives a work efficiency RPₐₗₗ of the target space by summing the work efficiency RP_{V} and the work efficiency RP_{T} using [symbol] .

15. The information processing apparatus according to claim 1, wherein
the communication unit communicates with an environment control apparatus which causes changes in an environmental state of the target space,
the generation unit generates, based on the comfort information, an environment control instruction for controlling the environment control apparatus which causes the changes in the environmental state of the target space, and
the communication unit transmits the environment control instruction to the environment control apparatus.

16. The information processing apparatus according to claim 15, wherein
when the comfort index is smaller than a threshold, the generation unit generates the environment control instruction for the environment control apparatus to cause the changes in the environmental state of the target space such that the comfort index becomes equal to or greater than the threshold.

17. An information processing system comprising:
the information processing apparatus according to claim 1; and
at least one of sensors, each being identical to the sensor.

18. An information processing system comprising:
the information processing apparatus according to claim 10; and
at least one of sensors, each being identical to the sensor.

19. An information processing method comprising:
receiving, by an information processing apparatus, statistical information from a sensor, the sensor having a measuring unit which measures environment information regarding environment in a target space and a generation unit which generates, based on the environment information, the statistical information regarding the environment of the target space;
deriving, by the information processing apparatus and based on the statistical information, a comfort index representing a comfort level of the target space according to a predetermined derivation method;
generating, by the information processing apparatus, comfort information representing the comfort index of the target space; and
authenticating, by the information processing apparatus and based on the statistical information, that the statistical information is information regarding the target space.

20. A computer readable medium having stored thereon a program that causes a computer to function as:
a communication unit which receives statistical information from a sensor, the sensor having a measuring unit which measures environment information regarding environment in a target space and a generation unit which generates, based on the environment information, the statistical information regarding the environment of the target space;
a derivation unit which derives, based on the statistical information, a comfort index representing a comfort level of the target space according to a predetermined derivation method;
a generation unit which generates comfort information representing the comfort index of the target space; and
an authentication unit which authenticates, based on the statistical information, that the statistical information is information regarding the target space.
